(19) United States
(12) Patent Application Publication
(10) Pub. No.: US 2010/0144761 A1
(43) Pub. Date: Jun. 10, 2010

(54) Antimicrobial Molecules For Treating Multi-Drug Resistant And Extensively Drug Resistant Strains Of Mycobacterium

(75) Inventors: Richard W. Scott, Radnor, PA (US); Dahui Liu, Wynnewood, PA (US)

Correspondence Address: Pepper Hamilton LLP, 400 Berwyn Park, 899 Cassatt Road, Berwyn, PA, 19312-1183, US

(73) Assignee: POLYMEDIX, INC.

(21) Appl. No.: 12/633,971

(22) Filed: Dec. 9, 2009

Related U.S. Application Data
(60) Provisional application No. 61/121,295, filed on Dec. 10, 2008.

(51) Int. Cl. A01N 43/54; A01N 37/18; A61K 31/17

(52) U.S. Cl. 514/256; 514/616; 514/597

(57) ABSTRACT

The present invention provides methods of inhibiting the growth of Mycobacterium species or treating an animal having a Mycobacterium infection (including multi-drug resistance strains and extensively drug resistant strains) by administering a compound of the invention, a salt thereof, or a composition comprising the same.

### FIELD OF THE INVENTION

The present invention is directed, in part, to methods of treating multi-drug resistant tuberculosis (MDR-TB) and extensively drug resistant tuberculosis (XDR-TB) with antimicrobial compounds and compositions.

### BACKGROUND OF THE INVENTION

Tuberculosis (TB) is a highly contagious disease that affects one-third of the world's population today. There are 8 million newly reported cases each year and 3.1 million people die from the disease annually. TB is the leading cause of death of women, AIDS patients, and the young in the world. There are more deaths from TB than any other single infectious disease. Worldwide, 30 to 50% of AIDS deaths are caused by TB. Globally, the population weighted mean of multi-drug resistant (MDR) TB among all TB cases is estimated at about 5%. Extensively-drug resistant (XDR) TB is more expensive and difficult to treat than MDR-TB and outcomes for XDR-TB patients are much worse. XDR-TB is widespread with 45 countries having reported at least one case (see, e.g. “Anti-Tuberculosis Drug Resistance in The World, Fourth Global Report: The WHO/IUATLD Global Project on Anti-tuberculosis, Drug Resistance Surveillance, 2002-2007”; World Health Organization Press, World Health Organization, 20 Avenue Appia, 1211 Geneva 27, Switzerland).

Mycobacterium tuberculosis (M. tuberculosis) is the primary infectious agent for TB, and drug resistance has become a paramount issue, accounting for over 50 million infections world wide. Several anti-infective agents have been identified that combat M. tuberculosis and other tuberculosis-causing organisms; the emergence of multi-drug resistant (MDR) and extensively-drug resistant (XDR) organisms, however, has severely limited their effectiveness. A current therapeutic strategy for active disease is to treat with multiple drugs for 6 to 9 months; a course of therapy that is difficult to manage for compliance, thereby exacerbating the development of resistance. Furthermore, many of the anti-TB agents interfere with HIV therapy creating a dangerous upward spiral in disease progression and severity in co-infected individuals.

Accordingly, there is need for anti-TB drugs that have properties such as having activity against MDR/XDR strains of M. tuberculosis, working in shorter duration of time in treatment, and/or not interfering with existing HIV therapies. The compounds, compositions comprising the compounds, and methods described herein help meet this and other needs.

### SUMMARY OF THE INVENTION

The present invention provides, in part, methods of inhibiting the growth of a Mycobacterium species comprising contacting the Mycobacterium species with an effective amount of a compound or salt thereof, wherein the compound or salt thereof is selected from:

a) a compound of Formula I:

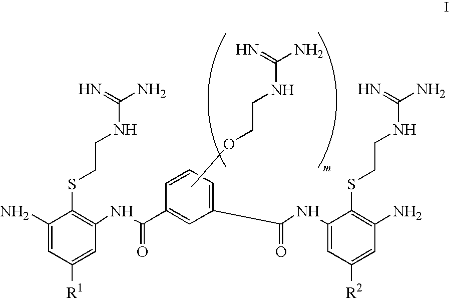

or salt thereof, wherein R¹ is H or C₁₋₁₀ alkyl; R² is H or C₁₋₁₀ alkyl; and m is 1 or 2; b) a compound of Formula II:

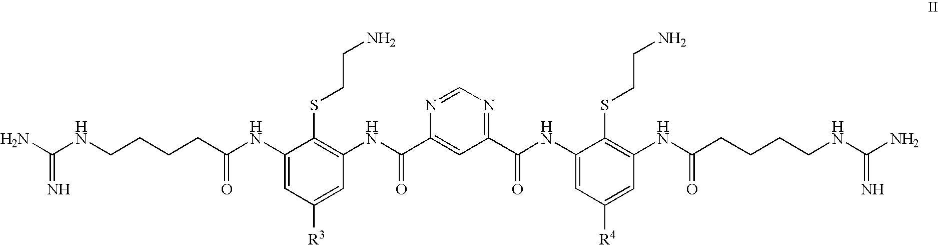

or salt thereof, wherein R³ is H or C₁₋₁₀ alkyl; and R⁴ is H or C₁₋₁₀ alkyl; and c) a compound of Formula III:

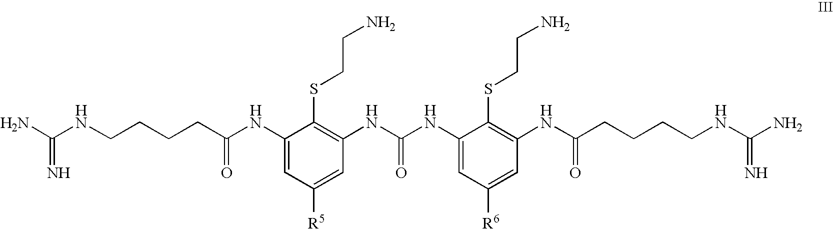

or salt thereof, wherein R⁵ is H or C₁₋₁₀ alkyl; and R⁶ is H or C₁₋₁₀ alkyl.

In some embodiments, the compound or salt thereof is a compound of Formula I or salt thereof. In some embodiments, the compound of Formula I or salt thereof is a compound of Formula Ia:

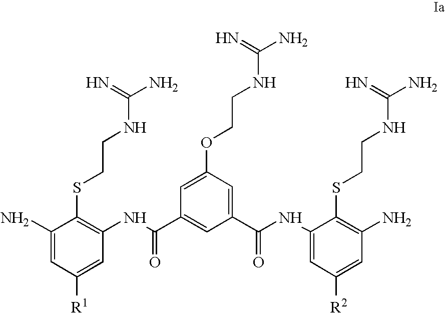

or salt thereof, wherein R¹ is H or C₁₋₈ alkyl; and R² is H or C₁₋₈ alkyl. In some embodiments, R¹ and R² are each, independently, C₁₋₈ alkyl. In some embodiments, R¹ and R² are each, independently, propan-2-yl, 2-methylpropan-2-yl, 2-methylbutan-2-yl, 2,3-dimethylbutan-2-yl, or 2,3,3-trimethylbutan-2-yl. In some embodiments, R¹ and R² are the same. In some embodiments, R¹ and R² are each 2-methylpropan-2-yl. In some embodiments, the compound of Formula I or salt thereof is a compound of Formula Ib:

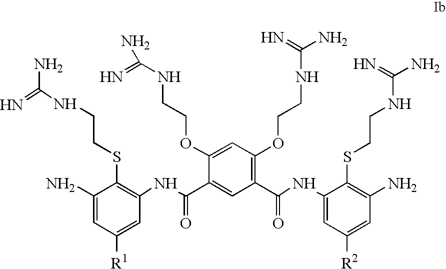

or salt thereof, wherein R¹ is H or C₁₋₁₀ alkyl; and R² is H or C₁₋₁₀ alkyl. In some embodiments, R¹ and R² are each, independently, H or C₁₋₈ alkyl. In some embodiments, R¹ and R² are each, independently, propan-2-yl, 2-methylpropan-2-yl, 2-methylbutan-2-yl, 2,3-dimethylbutan-2-yl, or 2,3,3-trimethylbutan-2-yl. In some embodiments, R¹ and R² are the same. In some embodiments, R¹ and R² are each 2-methylpropan-2-yl.

In some embodiments, the compound or salt thereof is a compound of Formula II or salt thereof. In some embodiments, R³ and R⁴ are each, independently, H or C₁₋₈ alkyl. In some embodiments, R³ and R⁴ are the same. In some embodiments, R³ and R⁴ are each H.

In some embodiments, the compound or salt thereof is a compound of Formula III or salt thereof. In some embodiments, R⁵ and R⁶ are each, independently, H or C₁₋₈ alkyl. In some embodiments, R⁵ and R⁶ are the same. In some embodiments, R⁵ and R⁶ are each 2-methylpropan-2-yl.

In some embodiments, the compound or salt thereof is a compound selected from:

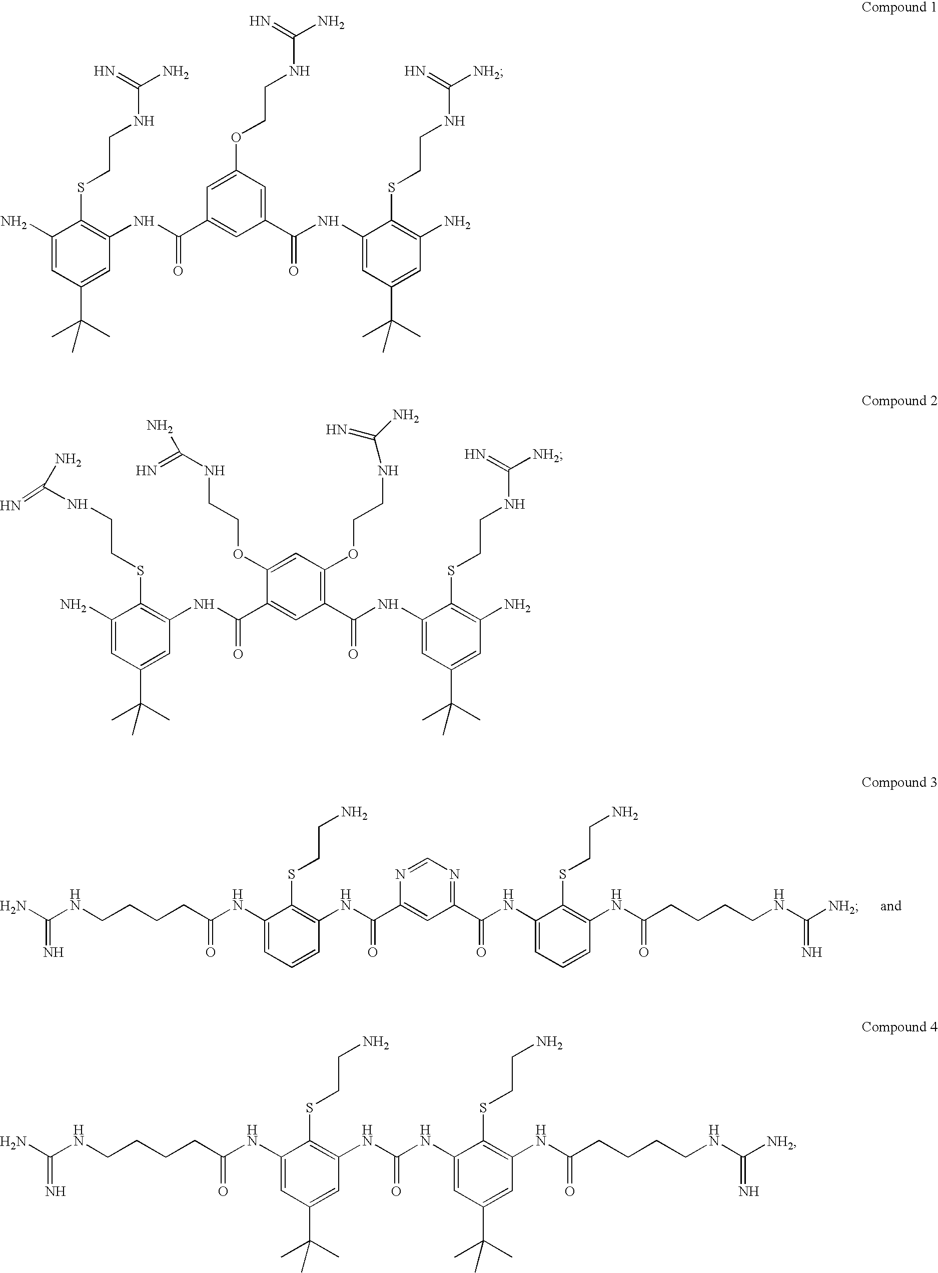

or a salt thereof.

In some embodiments, the Mycobacterium species is Mycobacterium Tuberculosis. In some embodiments, the Mycobacterium Tuberculosis is a multi-drug resistant strain. In some embodiments, the Mycobacterium Tuberculosis is an extensively drug resistant strain.

The present invention also provides, in part, methods of treating an animal having a Mycobacterium infection comprising administering to the animal a therapeutically effective amount of a compound or a pharmaceutically acceptable salt thereof, wherein the compound or pharmaceutically acceptable salt thereof is selected from: a) a compound of Formula I:

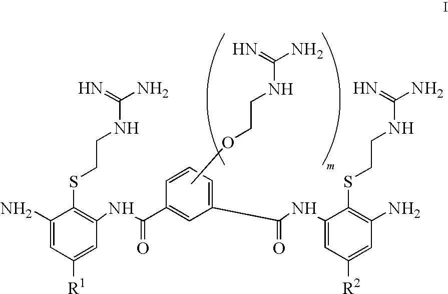

or a pharmaceutically acceptable salt thereof, wherein R¹ is H or C₁₋₁₀ alkyl; R² is H or C₁₋₁₀ alkyl; and m is 1 or 2; b) a compound of Formula II:

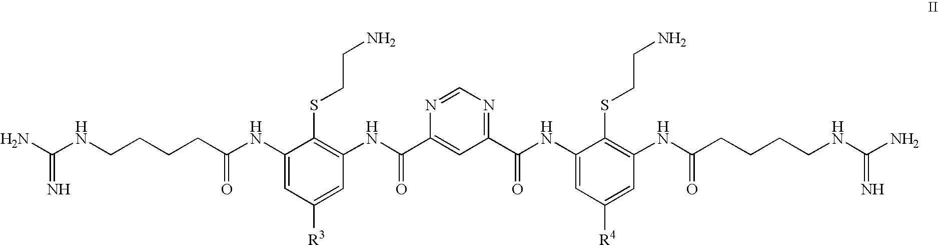

or a pharmaceutically acceptable salt thereof, wherein R³ is H or C₁₋₁₀ alkyl; and R⁴ is H or C₁₋₁₀ alkyl; and c) a compound of Formula III:

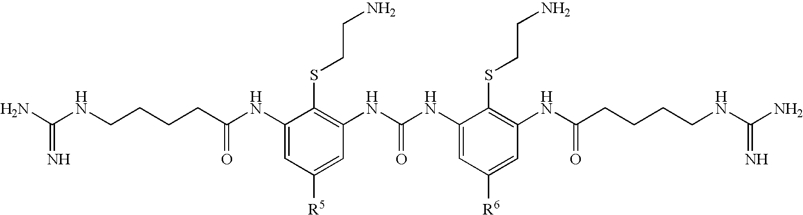

or a pharmaceutically acceptable salt thereof, wherein R⁵ is H or C₁₋₁₀ alkyl; and R⁶ is H or C₁₋₁₀ alkyl.

In some embodiments, the compound or pharmaceutically acceptable salt thereof is a compound of Formula I or a pharmaceutically acceptable salt thereof. In some embodiments, the compound of Formula I or pharmaceutically acceptable salt thereof is a compound of Formula Ia:

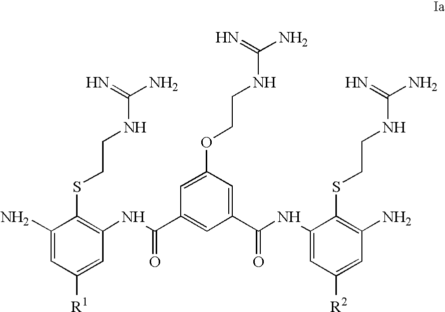

or a pharmaceutically acceptable salt thereof, wherein R¹ is H or C₁₋₈ alkyl; R² is H or C₁₋₈ alkyl. In some embodiments, R¹ and R² are each, independently, C₁₋₈ alkyl. In some embodiments, R¹ and R² are each, independently, propan-2-yl, 2-methylpropan-2-yl, 2-methylbutan-2-yl, 2,3-dimethylbutan-2-yl, or 2,3,3-trimethylbutan-2-yl. In some embodiments, R¹ and R² are the same. In some embodiments, R¹ and R² are each 2-methylpropan-2-yl. In some embodiments, the compound of Formula I or pharmaceutically acceptable salt thereof is a compound of Formula Ib:

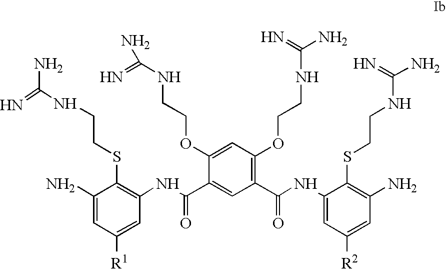

or pharmaceutically acceptable salt thereof, wherein R¹ is H or C₁₋₁₀ alkyl; and R² is H or C₁₋₁₀ alkyl. In some embodiments, R¹ and R² are each, independently, H or C₁₋₈ alkyl. In some embodiments, R¹ and R² are each, independently, propan-2-yl, 2-methylpropan-2-yl, 2-methylbutan-2-yl, 2,3-dimethylbutan-2-yl, or 2,3,3-trimethylbutan-2-yl. In some embodiments, R¹ and R² are the same. In some embodiments, R¹ and R² are each 2-methylpropan-2-yl.

In some embodiments, the compound or pharmaceutically acceptable salt thereof is a compound of Formula II or pharmaceutically acceptable salt thereof. In some embodiments, R³ and R⁴ are each, independently, H or C₁₋₈ alkyl. In some embodiments, R³ and R⁴ are the same. In some embodiments, R³ and R⁴ are each H.

In some embodiments, the compound or pharmaceutically acceptable salt thereof is a compound of Formula III or pharmaceutically acceptable salt thereof. In some embodiments, R⁵ and R⁶ are each, independently, H or C₁₋₈ alkyl. In some embodiments, R⁵ and R⁶ are the same. In some embodiments, R⁵ and R⁶ are each 2-methylpropan-2-yl.

In some embodiments, the compound or pharmaceutically acceptable salt thereof is a compound selected from:

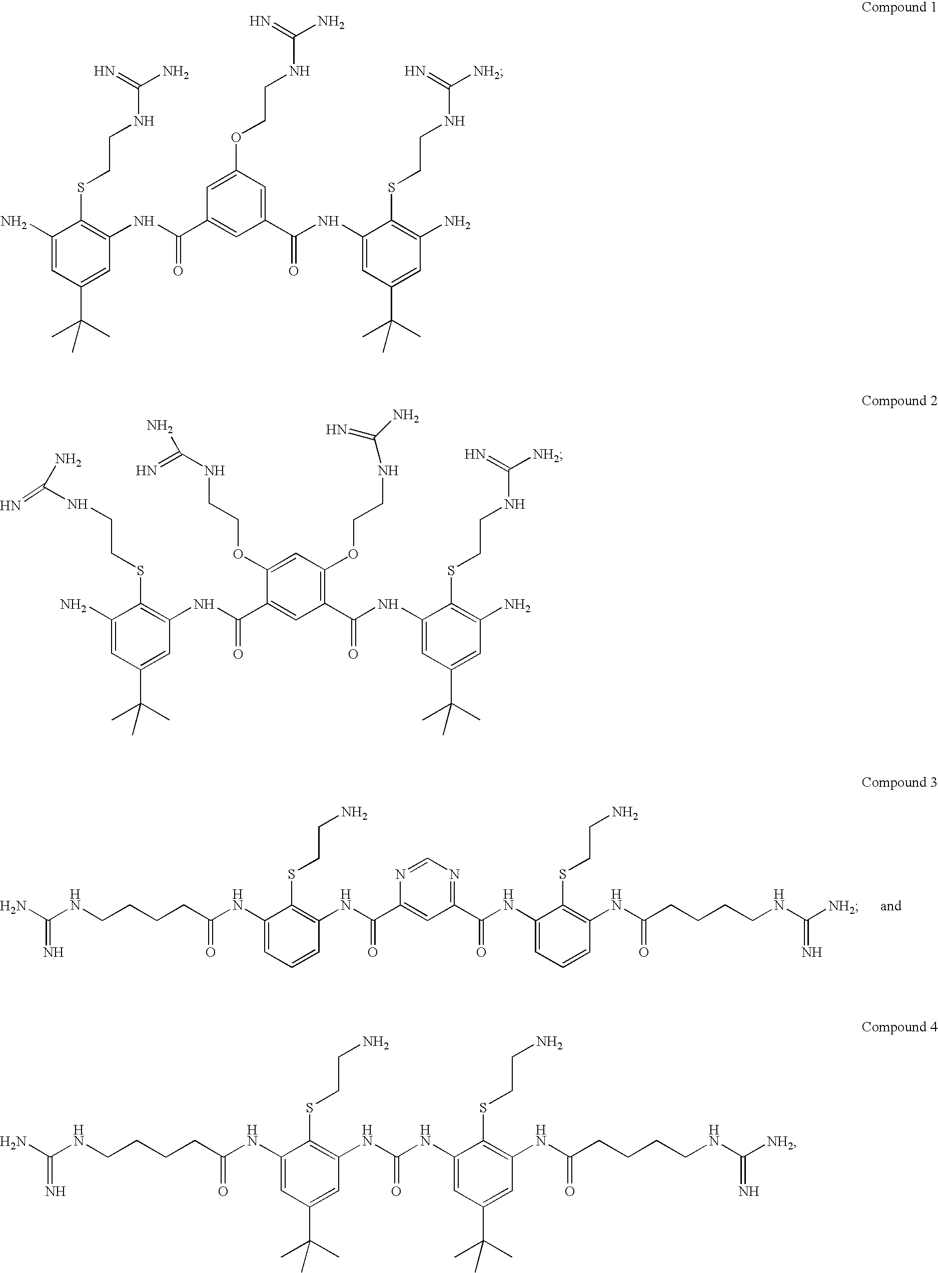

or a pharmaceutically acceptable salt thereof.

In some embodiments, the Mycobacterium infection is Mycobacterium Tuberculosis. In some embodiments, the Mycobacterium Tuberculosis is a multi-drug resistant strain. In some embodiments, the Mycobacterium Tuberculosis is an extensively drug resistant strain.

In some embodiments, in the methods described herein, the compound or salt thereof, or pharmaceutically acceptable salt thereof, is present in a pharmaceutical composition.

### DESCRIPTION OF EMBODIMENTS

As used herein, the term “about” means ±5% of the value it modifies.

As used herein, the term “alkyl” is meant to refer to a saturated hydrocarbon group which is straight-chained or branched. Examples of alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to 20, from 2 to 20, from 1 to 10, from 1 to 8, from 1 to 6, from 1 to 4, or from 1 to 3 carbon atoms. At various places in the present specification, substituents of compounds of the invention are disclosed in groups or in ranges. It is specifically intended that the invention include each and every individual subcombination of the members of such groups and ranges. For example, the term “C₁₋₈ alkyl” is specifically intended to individually disclose methyl, ethyl, C₃ alkyl, C₄ alkyl, C₅ alkyl, C₆ alkyl, C₇ alkyl, and C₈ alkyl, or any subgroup thereof.

As used herein, when an optionally multiple substituent is designated in the form:

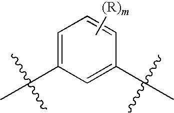

then it is understood that substituent R can occur m number of times on the ring, and R (if selected from a Markush group) can be a different moiety at each occurrence. Further, in the above example, any floating substituent such as R in the above example, can replace a hydrogen attached to one of the ring-forming carbon atoms.

As used herein, the phrase “inhibiting the growth” of a Mycobacterium species means reducing by any measurable amount the growth of one or more bacteria. In some embodiments, the inhibition of growth may result in cell death of the bacteria.

As used herein, the phrases “MDR-TB”, “multi-drug resistant TB”, and “multi-drug resistant Tuberculosis” mean TB with resistance to isoniazid and rifampicin, the two most powerful first line drugs.

As used herein, the phrases “XDR-TB”, “extensively drug resistant TB”, and “extensively drug resistant Tuberculosis” mean MDR-TB with resistance to any one of the fluoroquinolone drugs and to at least one of the following three injectable second-line drugs: amikacin, capreomycin, or kanamycin.

As used herein, the term “anti-TB” means that the compound inhibits, prevents, or destroys the growth or proliferation of a tuberculosis-causing organism (such as a Mycobacterium species).

As used herein, the term “animal” includes, but is not limited to, humans and non-human vertebrates such as mammals (e.g., mice, rats, other rodents, rabbits, dogs, cats, swine, cattle, sheep, horses, and primates).

As used herein, the term “substantially” means at least about 80%, at least about 90%, at least about 95%, or at least about 99%.

As used herein, the phrase “therapeutically effective amount” is an amount sufficient to decrease or inhibit growth of a Mycobacterium species.

The present invention provides compounds or salts thereof or pharmaceutically acceptable salts thereof, wherein the compounds, salts, or pharmaceutically acceptable salts thereof are selected from:

a) a compound of Formula I:

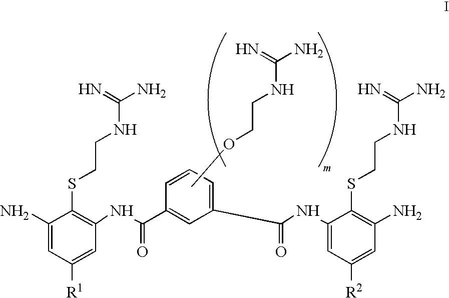

or salt or pharmaceutically acceptable salt thereof, wherein R¹ is H or C₁₋₁₀ alkyl; R² is H or C₁₋₁₀ alkyl; and m is 1 or 2;

b) a compound of Formula II:

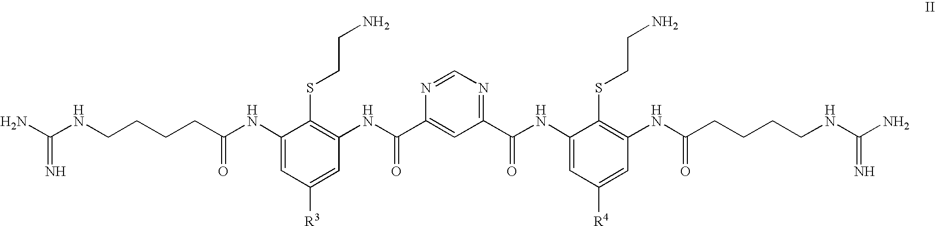

or salt or pharmaceutically acceptable salt thereof, wherein R³ is H or C₁₋₁₀ alkyl; and R⁴ is H or C₁₋₁₀ alkyl; and

c) a compound of Formula III:

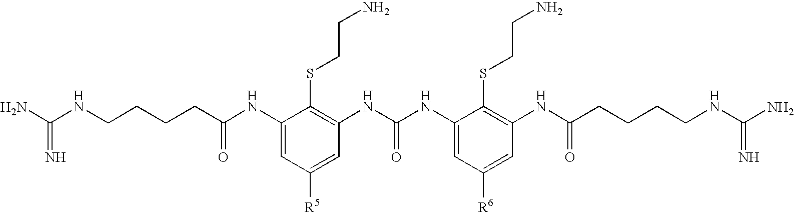

or salt or pharmaceutically acceptable salt thereof, wherein R⁵ is H or C₁₋₁₀ alkyl; and R⁶ is H or C₁₋₁₀ alkyl. Such compounds are synthetic, small molecule, non-peptidic mimics of host defense proteins (HDPs) that are designed to adopt amphiphilic conformations and/or exhibit potent antimicrobial activity while being non-toxic to host cells. These compounds may be advantageous because of their small size, which may increase stability and may enhance tissue distribution, and because of the ability to fine-tune their physical/chemical properties for optimization of potency and safety.

In some embodiments, the compounds or salts or pharmaceutically acceptable salts thereof are compounds of Formula I or salts or pharmaceutically acceptable salts thereof. In some embodiments, the compounds or salts or pharmaceutically acceptable salts thereof are compounds of Formula Ia:

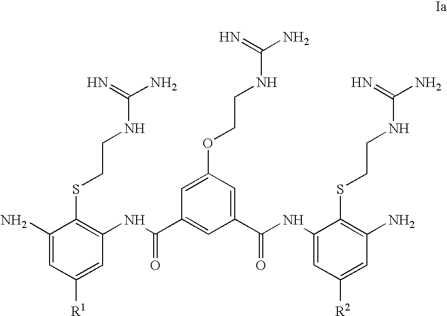

or salts or pharmaceutically acceptable salts thereof, wherein R¹ is H or C₁₋₈ alkyl; and R² is H or C₁₋₈ alkyl.

In some embodiments of the compounds of Formula Ia or salts or pharmaceutically acceptable salts thereof, R¹ and R² are each, independently, H or C₁₋₈ alkyl. In some embodiments, R¹ and R² are each, independently, C₁₋₈ alkyl, C₂₋₇ alkyl, C₃₋₇ alkyl, or C₃₋₆ alkyl. In some embodiments, R¹ and R² are each, independently, propan-2-yl, 2-methylpropan-2-yl, 2-methylbutan-2-yl, 2,3-dimethylbutan-2-yl, or 2,3,3-trimethylbutan-2-yl. In some embodiments, R¹ and R² are each, independently, branched C₃₋₇ alkyl or branched C₃₋₆ alkyl. In some embodiments, R¹ and R² are each, independently, H or C₁₋₄ alkyl. In some embodiments, R¹ and R² are each independently, H, methyl, ethyl, propan-1yl, propan-2-yl, butan-1-yl, butan-2-yl, or 2-methylpropan-2-yl. In some embodiments, R¹ and R² are each independently, H, methyl, or ethyl. In some embodiments, R¹ and R² are the same. In other embodiments, R¹ and R² are different. In some embodiments, R¹ and R² are each 2-methylpropan-2-yl.

In some embodiments, the compounds or salts or pharmaceutically acceptable salts thereof are compounds of Formula Ib:

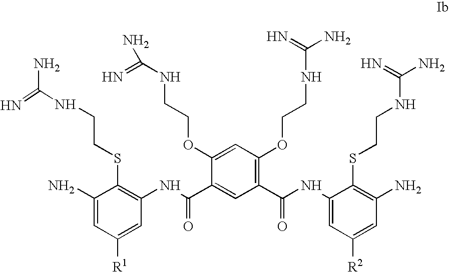

or salts or pharmaceutically acceptable salts thereof, wherein R¹ is H or C₁₋₈ alkyl; and R² is H or C₁₋₈ alkyl.

In some embodiments of the compounds of Formula Ib or salts or pharmaceutically acceptable salts thereof, R¹ and R² are each, independently, H or C₁₋₈ alkyl. In some embodiments, R¹ and R² are each, independently, C₁₋₈ alkyl, C₂₋₇ alkyl, C₃₋₇ alkyl, or C₃₋₆ alkyl. In some embodiments, R¹ and R² are each, independently, propan-2-yl, 2-methylpropan-2-yl, 2-methylbutan-2-yl, 2,3-dimethylbutan-2-yl, or 2,3,3-trimethylbutan-2-yl. In some embodiments, R¹ and R² are each, independently, branched C₃₋₇ alkyl or branched C₃₋₆ alkyl. In some embodiments, R¹ and R² are each, independently, H or C₁₋₄ alkyl. In some embodiments, R¹ and R² are each independently, H, methyl, ethyl, propan-1yl, propan-2-yl, butan-1-yl, butan-2-yl, or 2-methylpropan-2-yl. In some embodiments, R¹ and R² are each independently, H, methyl, or ethyl. In some embodiments, R¹ and R² are the same. In some embodiments, R¹ and R² are different. In some embodiments, R¹ and R² are each 2-methylpropan-2-yl.

In some embodiments, the compounds or salts or pharmaceutically acceptable salts thereof are compounds of Formula II:

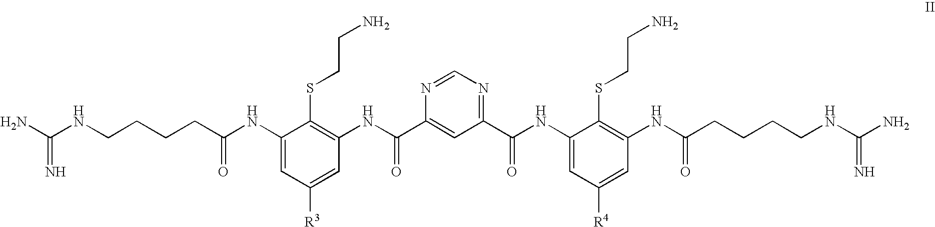

or salts or pharmaceutically acceptable salts thereof, wherein R³ is H or C₁₋₁₀ alkyl; and R⁴ is H or C₁₋₁₀ alkyl.

In some embodiments of the compounds of Formula II or salts or pharmaceutically acceptable salts thereof, R³ and R⁴ are each, independently, H or C₁₋₈ alkyl. In some embodiments, R³ and R⁴ are each, independently, H or C₁₋₄ alkyl. In some embodiments, R³ and R⁴ are each, independently, H, methyl, ethyl, propan-1yl, propan-2-yl, butan-1-yl, butan-2-yl, or 2-methylpropan-2-yl. In some embodiments, R³ and R⁴ are each, independently, H, methyl, or ethyl. In some embodiments, R³ and R⁴ are each, independently, C₁₋₈ alkyl, C₂₋₇ alkyl, C₃₋₇ alkyl, or C₃₋₆ alkyl. In some embodiments, R³ and R⁴ are each, independently, propan-2-yl, 2-methylpropan-2-yl, 2-methylbutan-2-yl, 2,3-dimethylbutan-2-yl, or 2,3,3-trimethylbutan-2-yl. In some embodiments, R³ and R⁴ are each, independently, branched C₃₋₇ alkyl or branched C₃₋₆ alkyl. In some embodiments, R³ and R⁴ are the same. In some embodiments, R³ and R⁴ are different. In some embodiments, R³ and R⁴ are each H.

In some embodiments, the compounds or salts or pharmaceutically acceptable salts thereof are compounds of Formula III:

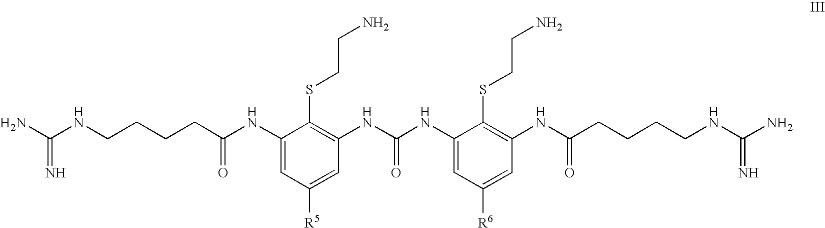

or salts or pharmaceutically acceptable salts thereof, wherein R⁵ is H or C₁₋₁₀ alkyl; and R⁶ is H or C₁₋₁₀ alkyl.

In some embodiments of the compounds of Formula III or salts or pharmaceutically acceptable salts thereof, R⁵ and R⁶ are each, independently, H or C₁₋₈ alkyl. In some embodiments, R⁵ and R⁶ are each, independently, C₁₋₈ alkyl, C₂₋₇ alkyl, C₃₋₇ alkyl, or C₃₋₆ alkyl. In some embodiments, R⁵ and R⁶ are each, independently, propan-2-yl, 2-methylpropan-2-yl, 2-methylbutan-2-yl, 2,3-dimethylbutan-2-yl, or 2,3,3-trimethylbutan-2-yl. In some embodiments, R⁵ and R⁶ are each, independently, branched C₃₋₇ alkyl or branched C₃₋₆ alkyl. In some embodiments, R⁵ and R⁶ are each, independently, H or C₁₋₄ alkyl. In some embodiments, R⁵ and R⁶ are each independently, H, methyl, ethyl, propan-1yl, propan-2-yl, butan-1-yl, butan-2-yl, or 2-methylpropan-2-yl. In some embodiments, R⁵ and R⁶ are each independently, H, methyl, or ethyl. In some embodiments, R⁵ and R⁶ are the same. In some embodiments, R⁵ and R⁶ are different. In some embodiments, R⁵ and R⁶ are each 2-methylpropan-2-yl.

In some embodiments, the compounds or salts or pharmaceutically acceptable salts thereof are compounds selected from:

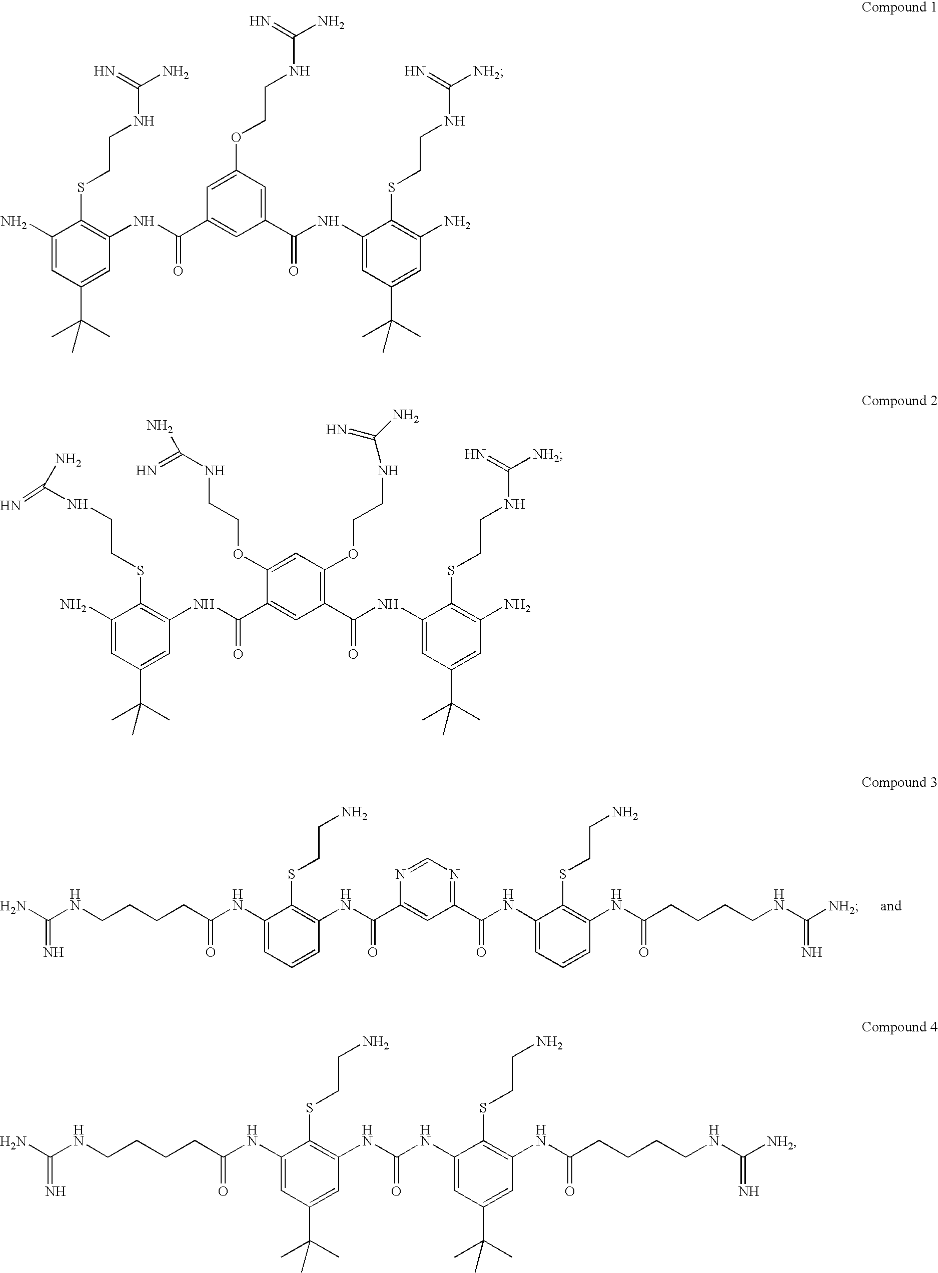

or salts or pharmaceutically acceptable salts thereof.

In some embodiments, the compounds or salts or pharmaceutically acceptable salts thereof are compounds selected from Compound 1, Compound 2, and Compound 3, or salts or pharmaceutically acceptable salts thereof.

In some embodiments, the compounds in the present invention can be chosen from one or more of the compounds (e.g., genuses, sub-genuses, and species) disclosed in U.S. Patent Application Publication Nos. US 2006/0041023 and/or US 2006/0241052, each of which is incorporated herein by reference in its entirety. The methods described herein can also be carried out using compounds disclosed as a genus, sub-genus, or species of U.S. Patent Application Publication Nos. US 2006/0041023 and/or US 2006/0241052.

Some of the compounds described herein may be capable of adopting amphiphilic conformations that allow for the segregation of polar and nonpolar regions of the molecule into different spatial regions. For example, some compounds of the invention may adopt amphiphilic conformations that are capable of disrupting the integrity of the cell membrane of microorganisms resulting in, for example, inhibition of growth of, for example, Mycobacterium species.

Although compounds disclosed herein are suitable, other functional groups can be incorporated into the compound with an expectation of similar results. For example, the distance between aromatic rings can impact the geometrical pattern of the compound and this distance can be altered by incorporating aliphatic chains of varying length, which can be optionally substituted or can comprise an amino acid, a dicarboxylic acid or a diamine. The distance between and the relative orientation of monomeric units within the compounds can also be altered by replacing the amide bond with a surrogate having additional atoms. Thus, replacing a carbonyl group with a dicarbonyl alters the distance between the monomeric units and the propensity of dicarbonyl unit to adopt an anti arrangement of the two carbonyl moieties and alter the periodicity of the compound. Pyromellitic anhydride represents an alternative to simple amide linkages which can alter the conformation and physical properties of the compound. Modern methods of solid phase organic chemistry (E. Atherton and R. C. Sheppard, Solid Phase Peptide Synthesis A Practical Approach IRL Press Oxford 1989) now allow the synthesis of homodisperse compounds with molecular weights approaching 5,000 Daltons. Other substitution patterns are equally effective. In addition, the compounds described herein can have O substituted for S, and S substituted for O, independently at each position.

The compounds described herein can be incorporated into compositions such as, for example, polishes, paints, sprays, or detergents formulated for application to a surface to inhibit the growth of a Mycobacterium species thereon. These surfaces include, but are not limited to, countertops, desks, chairs, laboratory benches, tables, floors, bed stands, tools, equipment, doorknobs, windows, and the like. The compounds described herein can also be incorporated into soaps and hand lotions. The present compositions, including the cleansers, polishes, paints, sprays, soaps, and detergents, can contain one or more of the compounds described herein. In addition, the compositions can optionally contain one or more of each of the following: solvents, carriers, thickeners, pigments, fragrances, deodorizers, emulsifiers, surfactants, wetting agents, waxes, and/or oils. For example, in some embodiments, the compounds can be incorporated into a formulation for external use as a pharmaceutically acceptable skin cleanser, particularly for the surfaces of human hands. Cleansers, polishes, paints, sprays, soaps, hand lotions, and detergents and the like containing the compounds described herein can be useful in homes and institutions, particularly but not exclusively, in hospital settings for the prevention of nosocomial infections.

In some embodiments, the compounds described herein can include derivatives referred to as prodrugs. The expression “prodrug” denotes a derivative of a known direct acting drug, which derivative has enhanced delivery characteristics and therapeutic value as compared to the drug, and is transformed into the active drug by an enzymatic or chemical process.

It is understood that the present invention encompasses the use, where applicable, of stereoisomers, diastereomers and optical isomers of the compounds described herein, as well as mixtures thereof, for the methods described herein. Additionally, it is understood that stereoisomers, diastereomers, and optical isomers of the compounds described herein, and mixtures thereof, are within the scope of the invention. By way of a non-limiting example, the mixture may be a racemate or the mixture may comprise unequal proportions of one particular stereoisomer over the other. Additionally, the compounds described herein can be provided as a substantially pure stereoisomer, diastereomer, or optical isomer.

In some embodiments, the compounds described herein can be provided in the form of an acceptable salt (i.e., a salt or a pharmaceutically acceptable salt). Salts can be provided for pharmaceutical use, or as an intermediate in preparing the pharmaceutically desired form of the compounds described herein. One salt that can be considered to be acceptable is the hydrochloride acid addition salt. Hydrochloride acid addition salts are often acceptable salts when the pharmaceutically active agent has an amine group that can be protonated. Since compounds described herein can be polyionic, such as a polyamine, the acceptable salt can be provided in the form of a poly(amine hydrochloride).

Polyamides that are useful for the present invention can be prepared by typical condensation polymerization and addition polymerization processes (see, for example, G. Odian, Principles of Polymerization, John Wiley & Sons, Third Edition (1991), M. Steven, Polymer Chemistry, Oxford University Press (1999)). Most commonly, the polyamides are prepared by a) thermal dehydration of amine salts of carboxylic acids, b) reaction of acid chlorides with amines and c) aminolysis of esters. Methods a) and c) may be of limited use in polymerizations of aniline derivatives which are generally prepared utilizing acid chlorides. The skilled chemist, however, will recognize that there are many alternative active acylating agents, for example phosphoryl anhydrides, active esters or azides, which may replace an acid chloride and which, depending of the particular polymer being prepared, may be superior to an acid chloride. The acid chloride route is likely the most versatile and has been used extensively for the synthesis of aromatic polyamides.

An alternative embodiment of the present invention is the corresponding polysulfonamides that can be prepared in analogous fashion by substituting sulfonyl chlorides for carboxylic acid chlorides.

Synthesis of compounds described herein can be carried out by routine and/or known methods such as those disclosed in, for example, U.S. Patent Application Publication Nos. US 2006/0041023 and/or US 2006/0241052, each of which is incorporated herein by reference in its entirety. Numerous pathways are available to incorporate polar and nonpolar side chains. Phenolic groups on the monomeric unit can be alkylated. Alkylation of the commercially available phenol can be accomplished with standard Williamson ether synthesis for the non-polar side chain with, for example, ethyl bromide as the alkylating agent. Polar sidechains can be introduced with bifunctional alkylating agents such as, for example, BOC—NH(CH₂)₂Br. Alternately, the phenol group can be alkylated to install the desired polar side chain function by employing the Mitsonobu reaction with, for example, BOC—NH(CH₂)₂—OH, triphenyl phosphine, and diethyl acetylenedicarboxylate. Standard conditions for reduction of the nitro groups and hydrolysis of the ester afford the amino acid. With the aniline and benzoic acid in hand, coupling can be effected under a variety of conditions. Alternately, the hydroxy group of the (di)nitrophenol can be converted to a leaving group and a functionality introduced under nucleophilic aromatic substitution conditions. Other potential scaffolds that can be prepared with similar sequences are methyl 2-nitro-4-hydroxybenzoate and methyl 2-hydroxy-4-nitrobenzoate.

The compounds described herein can also be designed using computer-aided computational techniques, such as de novo design techniques, to embody the amphiphilic properties. In general, de novo design of compounds is performed by defining a three-dimensional framework of the backbone assembled from a repeating sequence of monomers using molecular dynamics and quantum force field calculations. Next, side groups are computationally grafted onto the backbone to maximize diversity and maintain drug-like properties. The best combinations of functional groups are then computationally selected to produce a cationic, amphiphilic structures. Representative compounds can be synthesized from this selected library to verify structures and test their biological activity. Novel molecular dynamic and coarse grain modeling programs have also been developed for this approach because existing force fields developed for biological molecules, such as peptides, were unreliable in these oligomer applications (Car et al., Phys. Rev. Lett., 1985, 55, 2471-2474; Siepmann et al., Mol. Phys., 1992, 75, 59-70; Martin et al., J. Phys. Chem., 1999, B 103, 4508-4517; and Brooks et al., J. Comp. Chem., 1983, 4, 187-217). Several chemical structural series of compounds have been prepared. See, for example, WO 02/100295 A2, which is incorporated herein by reference in its entirety. The compounds described herein can be prepared in a similar manner. Molecular dynamic and coarse grain modeling programs can be used for a design approach. See, for example, U.S. patent application Ser. No. 10/446,171, filed May 28, 2003, and U.S. patent application Ser. No. 10/459,698, filed Jun. 12, 2003, each of which is incorporated herein by reference in its entirety.

After verifying the suitability of the force field by comparing computed predictions of the structure and thermodynamic properties to molecules that have similar torsional patterns and for which experimental data are available, the fitted torsions can then be combined with bond stretching, bending, one-four, van der Waals, and electrostatic potentials borrowed from the CHARMM (Brooks et al., J. Comp. Chem., 1983, 4, 187-217) and TraPPE (Martin et al., J. Phys. Chem., 1999, B 103, 4508-4517; Wick et al., J. Phys. Chem., 2000, B 104, 3093-3104) molecular dynamics force fields. To identify conformations that can adopt periodic folding patterns with polar groups and apolar groups lined up on the opposite sides, initial structures can be obtained with the Gaussian package (Frisch et al., Gaussian 98 (revision A.7) Gaussian Inc., Pittsburgh, Pa. 1998). Then, the parallelized plane-wave Car-Parrinello CP-MD (Car et al., Phys. Rev. Lett., 1985, 55, 2471-2474) program, (cf. Röthlisberger et al., J. Chem. Phys., 1996, 3692-3700) can be used to obtain energies at the minimum and constrained geometries. The conformations of the compounds without side-chains can be investigated in the gas phase. Both MD and MC methods can be used to sample the conformations. The former is useful for global motions of the compound. With biasing techniques (Siepmann et al., Mol. Phys., 1992, 75, 59-70; Martin et al., J. Phys. Chem., 1999, B 103, 4508-4517; Vlugt et al., Mol. Phys., 1998, 94, 727-733), the latter allows efficient sampling for compounds with multiple local minimum configurations that are separated by relatively large barriers.

The potential conformations are examined for positions to attach pendant groups that will impart amphiphilic character to the secondary structure. Compounds selected from the gas phase studies with suitable backbone conformations and with side-chains at the optimal positions to introduce amphiphilicity can be further evaluated in a model interfacial system. n-hexane/water can be chosen because it is simple and cheap for calculations while it mimics well the lipid/water bilayer environment. Compound secondary structures that require inter-compound interactions can be identified by repeating the above-mentioned calculations using a periodically repeated series of unit cells of various symmetries (so called variable cell molecular dynamics or Monte Carlo technique) with or without solvent. The results of these calculations can guide the selection of candidates for synthesis.

An example of the design, synthesis, and testing of arylamide polymers and oligomers is presented in, for example, Tew et al., Proc. Natl. Acad. Sci. USA, 2002, 99, 5110-5114, which is incorporated herein by reference in its entirety.

Compounds described herein can also be synthesized by solid-phase synthetic procedures well know to those of skill in the art. See, for example, Tew et al., Proc. Natl. Acad. Sci. USA, 2002, 99, 5110-5114; Barany et al., Int. J. Pept. Prot. Res., 1987, 30, 705-739; Solid-phase Synthesis: A Practical Guide, Kates, S. A., and Albericio, F., eds., Marcel Dekker, New York (2000); and Dorwald, F. Z., Organic Synthesis on Solid Phase: Supports, Linkers, Reactions, 2nd Ed., Wiley-VCH, Weinheim (2002).

The present invention also provides methods of inhibiting the growth of a Mycobacterium species comprising contacting the Mycobacterium species with an effective amount of a compound described herein, or salt or pharmaceutically acceptable salt thereof. In some embodiments, the compound is selected from Formula I (including Formula Ia and Formula Ib), Formula II, and Formula III.

Four of the compounds described herein have been tested and demonstrated to inhibit the growth of the H37Rv strain of M. tuberculosis in culture with a range of IC₉₀s of less than about 20 μM, about 10 μM, or about 5 μM (three of the compounds have IC₉₀s of less than about 5 μM), and to have low cytotoxicity to mammalian cells with a range of EC₅₀s greater than about 100 μM or about 300 μM. In some embodiments, the IC₉₀ of the compounds described herein (for M. tuberculosis H37Rv strain of M. tuberculosis) is less than about 10 μM or less than about 5 μM.

In some embodiments, some of the compounds described herein rapidly kill M. tuberculosis (for example in vitro). In some embodiments, some of the compounds described herein possess low cytotoxicity against mammalian cells. In some embodiments, the EC₅₀ of the compounds used in the present invention (for mammalian cells) is greater than about 200 μM or about 300 μM. In some embodiments, some of the compounds described herein have high selectivity against M. tuberculosis over mammalian cells. In some embodiments, the selective index (SI) values (the SI value is calculated by dividing the EC₅₀ by the IC₉₀) of some of the compounds described herein is greater than about 10, greater than about 20, greater than about 30, greater than about 40, greater than about 50, greater than about 60, greater than about 70, greater than about 80, greater than about 90, greater than about 100, greater than about 120, greater than about 150, or greater than about 200.

The present invention also provides methods of treating an animal having a Mycobacterium infection comprising administering to the animal a therapeutically effective amount of a compound or a pharmaceutically acceptable salt thereof. In some embodiments, the compound is selected from Formula I (including Formula Ia and Formula Ib), Formula II, and Formula III. In some embodiments, the Mycobacterium infection is caused by a Mycobacterium species, such as Mycobacterium tuberculosis. In some embodiments, the Mycobacterium species is active, dormant, or semi-dormant. In some embodiments, the active, dormant, or semi-dormant Mycobacterium species is not killed or inhibited by known TB drugs. In some embodiments, the Mycobacterium species is multi-drug resistant TB, with resistance to isoniazid and rifampicin. In some embodiments, the Mycobacterium species is extensively drug resistant TB, with resistance to any one of the fluoroquinolone drugs and to at least one of the following three injectable second-line drugs: amikacin, capreomycin, or kanamycin. In some embodiments, the Mycobacterium tuberculosis is multi-drug resistant TB, with resistance to isoniazid and rifampicin. In some embodiments, the Mycobacterium tuberculosis is extensively drug resistant TB, with resistance to any one of the fluoroquinolone drugs and to at least one of the following three injectable second-line drugs: amikacin, capreomycin, or kanamycin. In some embodiments, the methods described herein create or cause no new drug resistance. In some embodiments, the compound is present within a pharmaceutical composition.

In some embodiments, the animal being treated, such as a human, is “in need thereof” That is, the animal is in need of treatment. Thus, in some embodiments, the animal is treated for the purpose of treating the Mycobacterium infection. In some embodiments, the animal has been diagnosed with a Mycobacterium infection or is suspected of having a Mycobacterium infection. In some embodiments, the animal, or human, is in a population at risk of having a Mycobacterium infection, such as in a prison or hospital.

Those skilled in the art will recognize that the compounds described herein can be tested for anti-TB activity by methods well known to those of skill in the art (see, e.g., Collins et al., Antimicrobial Agents and Chemotherapy, 1997, 41, 1004-1009). Any compound found to be active can be purified to homogeneity and re-tested to obtain an accurate IC₉₀ or IC₅₀. Because these compounds can work by directly lysing bacterial cell membranes (rather than working on any specific receptor or intracellular target), the same mechanism utilized by the host defense proteins, drug resistance to these compounds is unlikely to develop. This premise is supported by experimental data showing that a negligible incidence of resistance development was observed in vitro in serial passage challenge assays using S. aureus. Thus, targeting bacterial cell membranes rather than any specific receptor or intracellular target represents a highly innovative and novel approach for treating TB (including MDR-TB and/or XRD-TB) and serves as one manner to distinguish the present invention from others in this field.

The compounds described herein can be administered in any conventional manner by any route where they are active. The compounds, or compositions thereof, can be administered to any body site or tissue. Administration can be systemic, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the compounds described herein (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams. The selection of the specific route of administration and the dose regimen is to be adjusted or titrated by the clinician according to methods known to the clinician to obtain the desired clinical response. The amount of compounds of the invention to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician). In some embodiments, suitable dosage ranges for intravenous (i.v.) administration are 0.01 mg to 500 mg per kg body weight, 0.1 mg to 100 mg per kg body weight, 1 mg to 50 mg per kg body weight, or 10 mg to 35 mg per kg body weight. Suitable dosage ranges for other modes of administration can be calculated based on the forgoing dosages as known by those skilled in the art. For example, recommended dosages for intradermal, intramuscular, intraperitoneal, subcutaneous, epidural, sublingual, intracerebral, intravaginal, transdermal administration or administration by inhalation are in the range of 0.001 mg to 200 mg per kg of body weight. Effective doses may be extrapolated from dose-response curves derived from in vitro or animal model test systems. Such animal models and systems are well known in the art.

The pharmaceutical compositions and/or formulations containing one or more of the compounds described herein and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder. The compositions comprise an effective amount of one or more of the compounds described herein. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives, and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compounds described herein can be formulated for parenteral administration by injection, such as, by bolus injection or continuous infusion. The compounds described herein can be administered by continuous infusion subcutaneously over a period of about 15 minutes to about 24 hours. Formulations for injection can be presented in unit dosage form, such as, in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compounds described herein can be formulated readily by combining these compounds with pharmaceutically acceptable carriers well known in the art. Such carriers help facilitate the compounds described herein to be formulated as tablets, pills, dragees, capsules, liquids, gels, syrups, slurries, suspensions and the like, for oral ingestion by a patient to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, for example, lactose, binders such as, for example, starches, and/or lubricants such as, for example, talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, for example, tablets or lozenges formulated in a conventional manner.

For administration by inhalation, the compounds described herein can be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base such as lactose or starch.

The compounds described herein can also be formulated in rectal compositions such as suppositories or retention enemas, for example, containing conventional suppository bases such as cocoa butter or other glycerides.

In addition to the formulations described above, the compounds described herein can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compounds described herein can be applied to, for example, a plaster, or can be applied by transdermal, therapeutic systems that are consequently supplied to the organism.

The pharmaceutical compositions of the compounds described herein can also comprise suitable solid or gel phase carriers or excipients. Examples of such carriers or excipients include, but are not limited to, calcium carbonate, calcium phosphate, various sugars, starches, cellulose derivatives, gelatin, and polymers such as, for example, polyethylene glycols.

In some embodiments, the compounds described herein can also be administered in combination (concurrently or serially) with other active ingredients such as antibiotics, including, but not limited to, fluoroquinolones, amikacin, capreomycin, or kanamycin.

In any of the methods described above and herein, the Mycobacterium species can be Mycobacterium tuberculosis. In some embodiments, the Mycobacterium species is active, dormant, or semi-dormant. In some embodiments, the active, dormant, or semi-dormant Mycobacterium species is not killed or inhibited by known TB drugs. In some embodiments, the Mycobacterium species is multi-drug resistant TB, with resistance to isoniazid and rifampicin. In some embodiments, the Mycobacterium species is extensively drug resistant TB, with resistance to any one of the fluoroquinolone drugs and to at least one of the following three injectable second-line drugs: amikacin, capreomycin, or kanamycin.

The present invention also provides pharmaceutical packs or kits comprising one or more containers filled with one or more compounds described herein. Optionally associated with such container(s) can be a notice in the form prescribed by a governmental agency regulating the manufacture, use or sale of pharmaceuticals or biological products, which notice reflects approval by the agency of manufacture, use or sale for human administration for treating a Mycobacterium infection. In some embodiments, the kit contains more than one compound described herein. In some embodiments, the kit comprises a compound described herein in a single injectable dosage form, such as a single dose within an injectable device such as a syringe with a needle.

The present invention also provides compounds described herein, or compositions or pharmaceutical compositions comprising the same, for use in preparation of a medicament for treating a Mycobacterium infection (including Mycobacterium tuberculosis, including MDR-TB and XDR-TB) in an animal and/or for inhibiting the growth of a Mycobacterium species. The present invention also provides compounds described herein, or compositions comprising the same, for treating a Mycobacterium infection (including Mycobacterium tuberculosis, including MDR-TB and XDR-TB) in an animal and/or for inhibiting the growth of a Mycobacterium species.

In order that the invention disclosed herein may be more efficiently understood, examples are provided below. It should be understood that these examples are for illustrative purposes only and are not to be construed as limiting the invention in any manner.

### EXAMPLES

### Example 1

### Susceptibility Assays Versus M. Tuberculosis (H37Rv Strain) and Cytotoxicity Assays versus Monkey VERO Cells (Actual Example)

To evaluate the effects of compounds of Formula I, II, and III on inhibiting the growth of a M. tuberculosis species, susceptibility assays of some compounds on M. tuberculosis (H37Rv strain) and cytotoxicity assays of some compounds on monkey VERO cells were performed.

The antimicrobial screen was conducted against the H37Rv strain of M. tuberculosis in BACTEC 12B medium using the Microplate Alamar Blue Assay (MABA) (see, e.g., Collins et al., Antimicrobial Agents and Chemotherapy, 1997, 41(5), 1004-1009). Compounds were tested in ten 2-fold dilutions to determine IC₉₀ values (an IC₉₀ value is defined as the concentration effecting a reduction in fluorescence of 90% relative to controls). Viability in the VERO cell cytotoxicity assay was measured after a 72 hour exposure using a luminescent cell viability assay that determines the number of viable cells based on quantitation of ATP. Cytotoxicity was determined using a curve fitting program to calculate EC₅₀ values. An SI (Selectivity Index) value was calculated by dividing the EC₅₀ by the IC₉₀.

The data of four screened compounds are provided in Table 1. Each of Compounds 1, 2, and 3 had an IC₉₀ value (v. M. tuberculosis) of less than 5 μM. Compound 4 had an IC₉₀ value (v. M. tuberculosis) of less than 20 μM. Each of Compounds 1, 2, and 4 had an ECₛₒ value (v. Monkey VERO Cells) of greater than 300 μM. Compound 3 had an EC₅₀ value (v. Monkey VERO Cells) of greater than 100 μM. Each of Compounds 1, 2, and 3 had an SI value greater than 20. Compound 4 had an SI value greater than 15.

TABLE 1

| | IC₉₀ (μg/mL) | | |
|---|---|---|---|
| | M. tuberculosis | EC₅₀ (μg/ml) | SI |
| Compound | (H37Rv strain) | VERO cells | (EC₅₀/IC₉₀) |
| Compound 1 | 2.2 | >300 | >136.4 |
| Compound 2 | 4.5 | >300 | >66.7 |
| Compound 3 | 3.6 | >100 | >27.8 |
| Compound 4 | 18.4 | >300 | >16.3 |

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A method of inhibiting the growth of a Mycobacterium species comprising contacting the Mycobacterium species with an effective amount of a compound or salt thereof, wherein the compound or salt thereof is selected from:
a) a compound of Formula I: 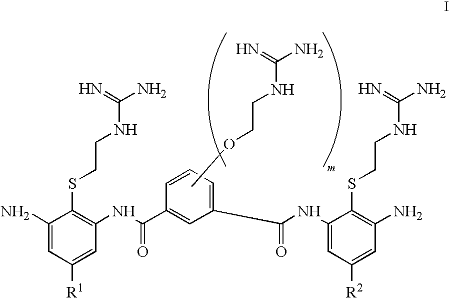
or salt thereof, wherein R¹ is H or C₁₋₁₀ alkyl; R² is H or C₁₋₁₀ alkyl; and m is 1 or 2;
b) a compound of Formula II: 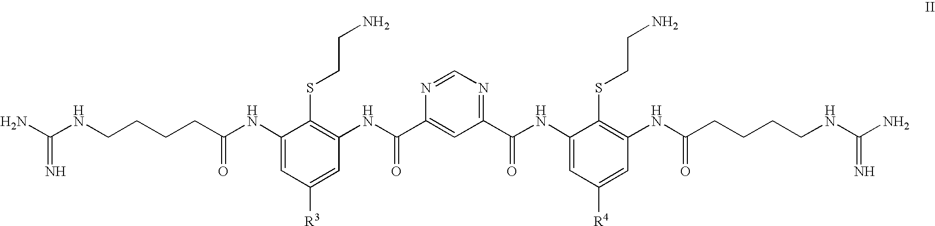
or salt thereof, wherein R³ is H or C₁₋₁₀ alkyl; and R⁴ is H or C₁₋₁₀ alkyl; and
c) a compound of Formula III: 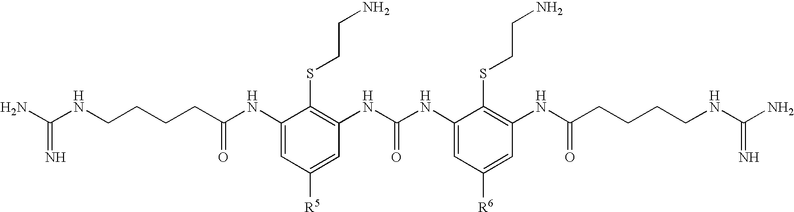
or salt thereof, wherein R⁵ is H or C₁₋₁₀ alkyl; and R⁶ is H or C₁₋₁₀ alkyl.

2. The method of claim 1 wherein the compound or salt thereof is a compound of Formula I or salt thereof.

3. The method of claim 2 wherein the compound of Formula I or salt thereof is a compound of Formula Ia: 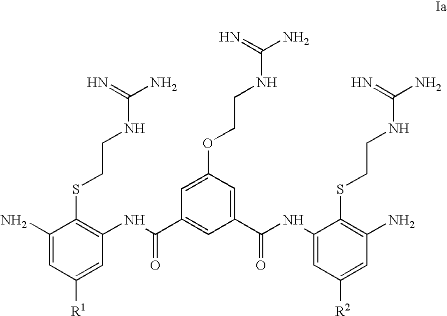
or salt thereof, wherein R¹ is H or C₁₋₈ alkyl; and R² is H or C₁₋₈ alkyl.

4-7. (canceled)

8. The method of claim 2 wherein the compound of Formula I or salt thereof is a compound of Formula Ib: 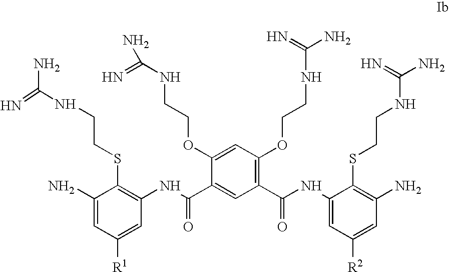
or salt thereof, wherein R¹ is H or C₁₋₁₀ alkyl; and R² is H or C₁₋₁₀ alkyl.

9-12. (canceled)

13. The method of claim 1 wherein the compound or salt thereof is a compound of Formula II or salt thereof.

14-16. (canceled)

17. The method of claim 1 wherein the compound or salt thereof is a compound of Formula III or salt thereof.

18-20. (canceled)

21. The method of claim 1 wherein the compound or salt thereof is a compound selected from: 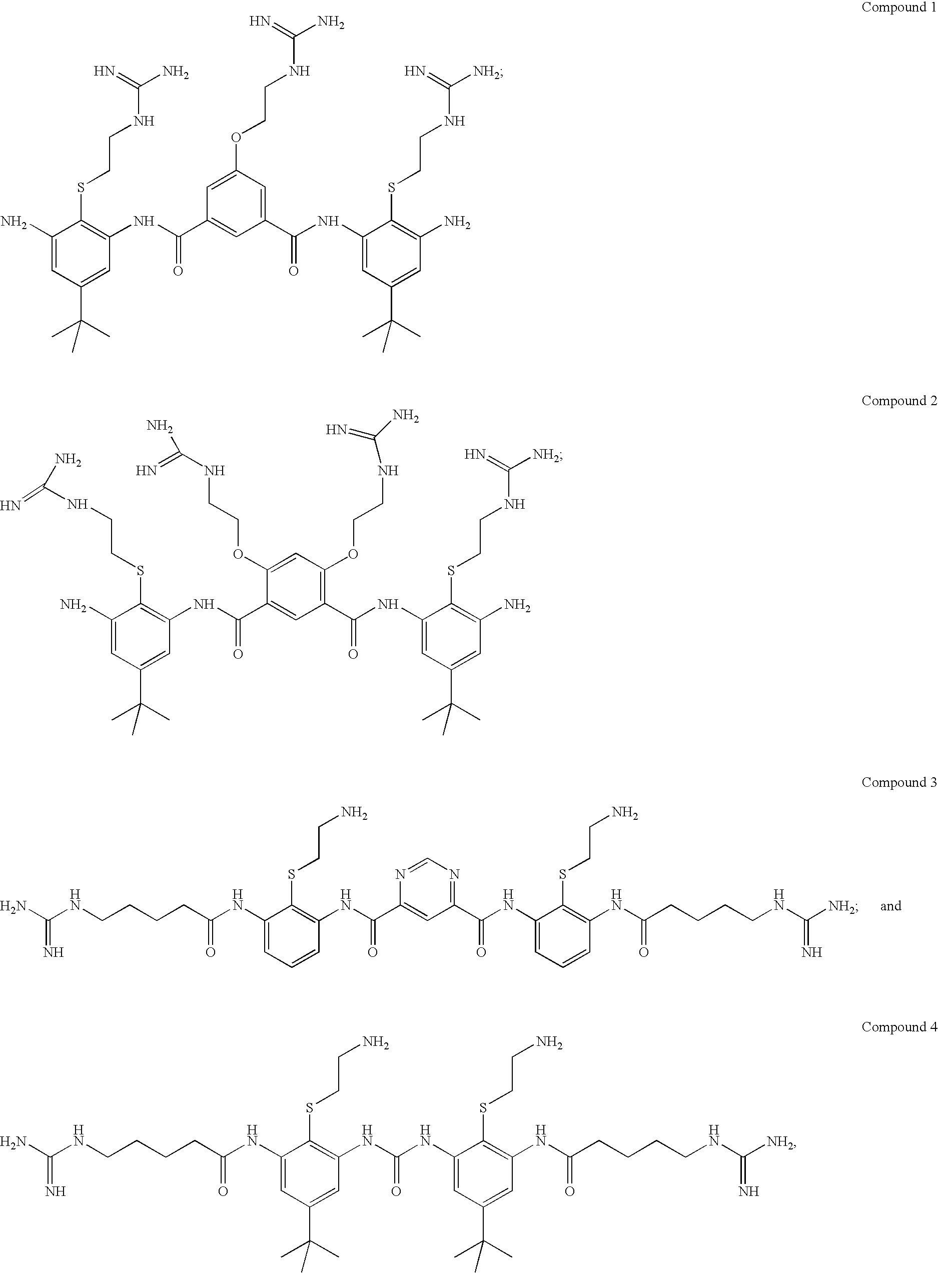
or a salt thereof.

22. The method of claim 1 wherein the Mycobacterium species is Mycobacterium Tuberculosis.

23. The method of claim 22 wherein the Mycobacterium Tuberculosis is a multi-drug resistant strain.

24. The method of claim 22 wherein the Mycobacterium Tuberculosis is an extensively drug resistant strain.

25. A method of treating an animal having a Mycobacterium infection comprising administering to the animal a therapeutically effective amount of a compound or a pharmaceutically acceptable salt thereof, wherein the compound or pharmaceutically acceptable salt thereof is selected from:
a) a compound of Formula I: 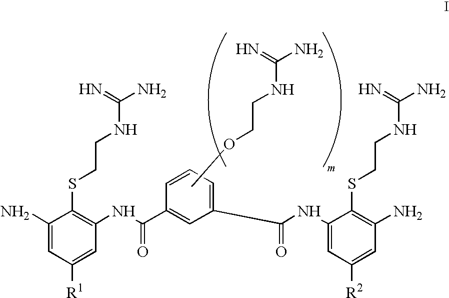
or a pharmaceutically acceptable salt thereof, wherein R¹ is H or C₁₋₁₀ alkyl; R² is H or C₁₋₁₀ alkyl; and m is 1 or 2;
b) a compound of Formula II: 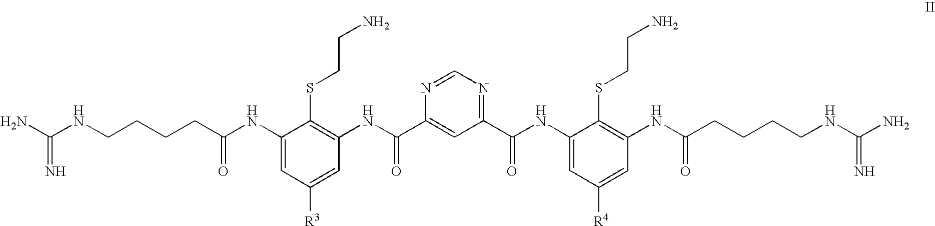
or a pharmaceutically acceptable salt thereof, wherein R³ is H or C₁₋₁₀ alkyl; and R⁴ is H or C₁₋₁₀ alkyl; and
c) a compound of Formula III: 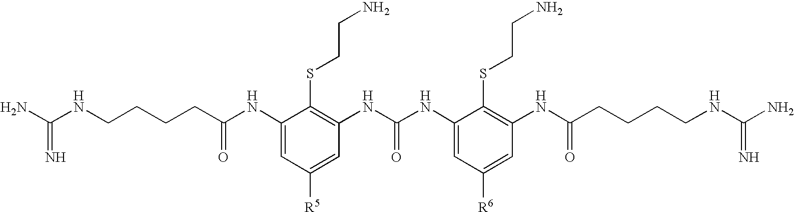
or a pharmaceutically acceptable salt thereof, wherein R⁵ is H or C₁₋₁₀ alkyl; and R⁶ is H or C₁₋₁₀ alkyl.

26. The method of claim 25 wherein the compound or pharmaceutically acceptable salt thereof is a compound of Formula I or a pharmaceutically acceptable salt thereof.

27. The method of claim 26 wherein the compound of Formula I or pharmaceutically acceptable salt thereof is a compound of Formula Ia: 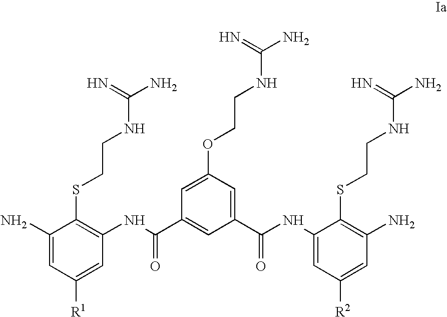
or a pharmaceutically acceptable salt thereof, wherein R¹ is H or C₁₋₈ alkyl; R² is H or C₁₋₈ alkyl.

28-31. (canceled)

32. The method of claim 26 wherein the compound of Formula I or pharmaceutically acceptable salt thereof is a compound of Formula Ib: 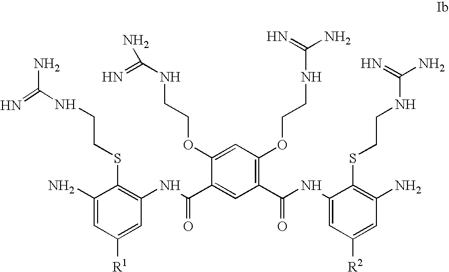
or pharmaceutically acceptable salt thereof, wherein R¹ is H or C₁₋₁₀ alkyl; and R² is H or C₁₋₁₀ alkyl.

33-36. (canceled)

37. The method of claim 25 wherein the compound or pharmaceutically acceptable salt thereof is a compound of Formula II or pharmaceutically acceptable salt thereof.

38-40. (canceled)

41. The method of claim 25 wherein the compound or pharmaceutically acceptable salt thereof is a compound of Formula III or pharmaceutically acceptable salt thereof.

42-44. (canceled)

45. The method of claim 25 wherein the compound or pharmaceutically acceptable salt thereof is a compound selected from: 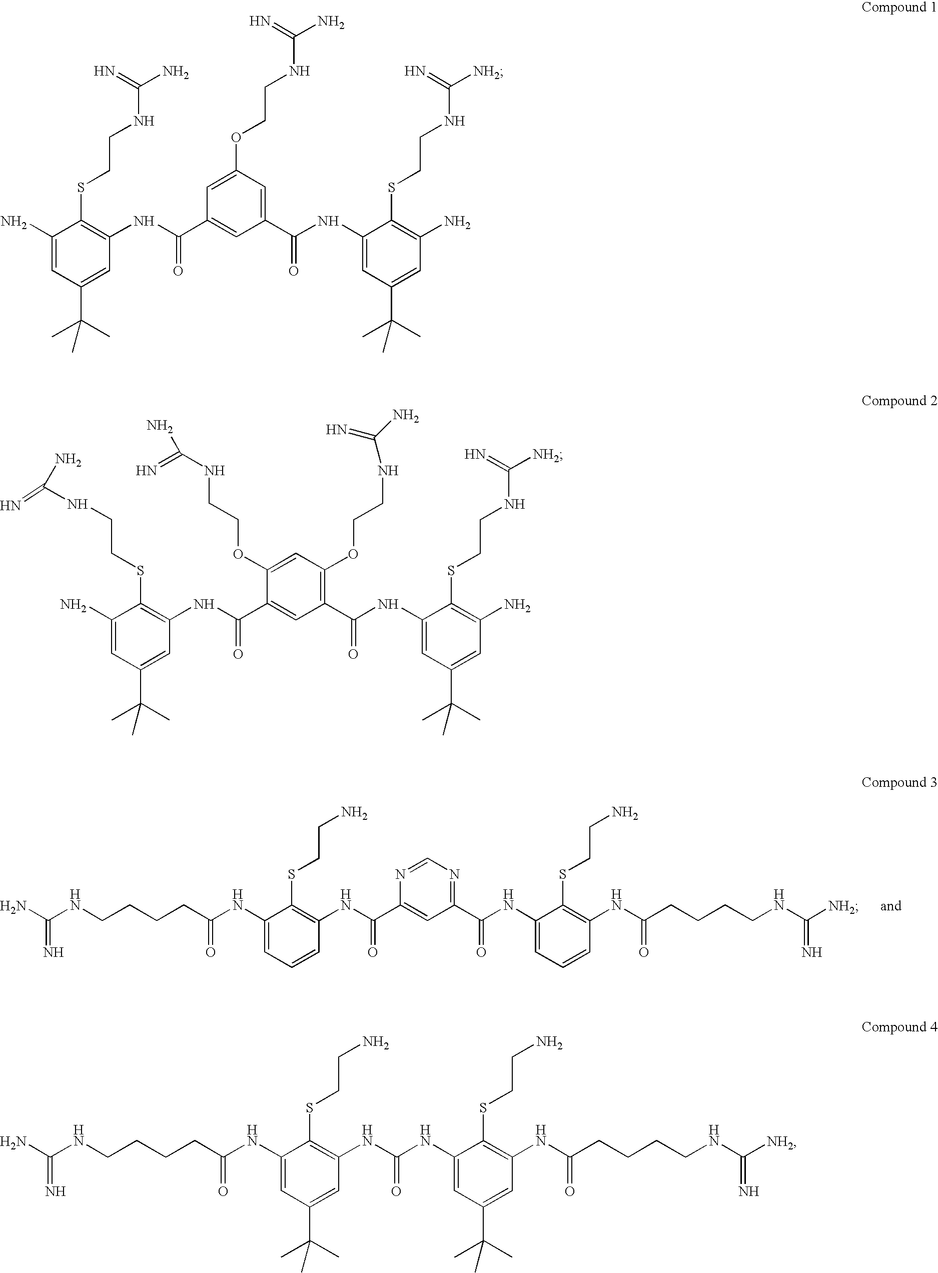
or a pharmaceutically acceptable salt thereof.

46. The method of claim 25 wherein the Mycobacterium infection is Mycobacterium Tuberculosis.

47. The method of claim 46 wherein the Mycobacterium Tuberculosis is a multi-drug resistant strain.

48. The method of claim 46 wherein the Mycobacterium Tuberculosis is an extensively drug resistant strain.

49. The method of claim 1 wherein the compound or salt thereof is present in a composition.

50-52. (canceled)
